(19) United States
(12) Patent Application Publication
(10) Pub. No.: US 2023/0135237 A1
(43) Pub. Date: May 4, 2023

(54) STRENGTH-ADJUSTABLE GUIDEWIRE

(71) Applicant: BARD PERIPHERAL VASCULAR, INC., Franklin Lakes, NJ (US)

(72) Inventors: Jianjiang Chen, Shanghai (CN); Chang-Keng Chen, Shanghai (CN)

(21) Appl. No.: 17/926,435

(22) PCT Filed: Apr. 16, 2021
(86) PCT No.: PCT/US2021/027735

§ 371 Date: Nov. 18, 2022

(51) Int. Cl. A61M 25/09

(52) U.S. Cl. CPC A61M 25/09; A61M 2025/09083; A61M 2025/09108; A61M 2025/09133; A61M 2025/09158

(57) ABSTRACT

A guidewire (10) is provided including a core wire (12), a resin layer (14) at least partially surrounding the core wire, and a coil wire (16) external to the resin layer. The resin layer extending over and along the core wire may comprise a series of windings, which may have a constant or variable pitch, or may comprise a sleeve. By applying a heat treatment to portions og the guidewire, the resin layer may be selectively melted to form a bond between the core wire and the coil wire. This technique may be used to provide the guidewire with a desired strength profile, without the need for tapering the ends of the core wire.

### BACKGROUND OF THE INVENTION

Balloon dilatation catheters are often used to treat lesions in vessels. Such catheters are typically guided to a treatment location in the vessel by a guidewire. Typically, the guidewire is a small diameter, highly flexible piece of a metal, such as stainless steel, sized to track easily through the vasculature from an access point and provide reliable guidance to the balloon catheter.

Past proposals for guidewires have been made, which typically include tapered ends and an outer coil to enhance flexibility. However, the past requirement for tapered ends on the core wire increases the cost and complexity of manufacturing the guidewire. This is because the core wire must be extensively processed prior to assembly.

Accordingly, a need is identified for a guidewire and a corresponding manner of manufacturing a guidewire that overcomes the foregoing limitations and others.

### SUMMARY OF THE INVENTION

An object of the disclosure is to provide a guidewire, which can be more efficiently manufactured at a lower cost, without suffering from a lack of flexibility.

According to one aspect of the disclosure, a guidewire includes a core wire, a resin layer at least partially surrounding the core wire, and a coil wire external to the resin layer.

In one embodiment, the core wire is not tapered. Thus, the core wire has a substantially constant diameter.

In this or other embodiments, the resin layer may comprise a spirally wound tape. This spirally wound tape may comprise windings having a generally fixed pitch. Alternatively, the spirally wound tape comprises windings having a variable pitch including first windings having a first spacing at a first end of the guidewire and second windings having a second spacing away from the first end of the guidewire. The resin layer may alternatively comprise a sleeve, and the guidewire may include welded ends.

The resin layer may be melted so as to form a bond between the core wire and the coil wire, such as for example at selected locations only.

According to a second aspect of the disclosure, a guidewire includes a core wire, a resin sleeve at least partially surrounding the core wire, and a coil wire external to the resin sleeve.

The core wire is not tapered, and may have a substantially constant diameter. The resin sleeve may be melted at selected locations so as to form a bond between the core wire and the coil wire.

According to yet another aspect of the disclosure, a guidewire comprising a core wire, a resin tape at least partially surrounding the core wire, and a coil wire external to the resin tape.

The core wire may have a substantially constant diameter, and thus is not tapered. The resin tape may be spirally wound, and may include windings having a fixed pitch. Alternatively, the spirally wound tape comprises windings having a variable pitch including first windings having a first spacing at a first end of the guidewire and second windings having a second spacing away from the first end of the guidewire.

The resin layer may be melted so as to form a bond between the core wire and the coil wire, such as at selected locations only.

This disclosure also relates to a method of forming a guidewire. The method includes providing a resin layer between an inner core wire and an outer coil wire, and applying heat to at least partially melt the resin layer and bond the inner core wire to the outer coil wire.

The providing step may comprise wrapping a resin tape around the inner core wire. The wrapping may be done at a variable pitch or a substantially constant pitch. The applying step may comprise applying the heat selectively to melt only portions of the resin layer. The providing step may also comprise providing a resin sleeve over the inner core wire.

### BRIEF DESCRIPTION OF THE DRAWINGS

The above and further advantages of the present disclosure may be better understood by referring to the following description in conjunction with the accompanying drawings in which:

FIG. 1 is a side view of a guidewire according to the disclosure;

FIG. 2 is an end view of the guidewire according to FIG. 1;

FIG. 3 is a side cross-sectional view of a first embodiment of the guidewire;

FIG. 4 is a side cross-sectional view of a first embodiment of the guidewire; and

FIG. 5 is a side cross-sectional view of a first embodiment of the guidewire.

The dimensions of some of the elements may be exaggerated relative to other elements for clarity or several physical components may be included in one functional block or element. Further, sometimes reference numerals may be repeated among the drawings to indicate corresponding or analogous elements. Moreover, some of the blocks depicted in the drawings may be combined into a single function.

### DETAILED DESCRIPTION

In the following detailed description, numerous specific details are set forth to provide a thorough understanding of the presently disclosed invention(s). The disclosed embodiments may be practiced without these specific details. In other instances, well-known methods, procedures, components, or structures may not have been described in detail so as not to obscure the present inventive concepts.

The invention is not limited in its application to the details of construction and the arrangement of the components set forth in the following description or illustrated in the drawings. The inventive concepts disclosed are capable of other embodiments or of being practiced or carried out in various ways. Also, it is to be understood that the phraseology and terminology employed herein are for the purpose of description and should not be regarded as limiting.

Certain features of the disclosed embodiments that are, for clarity, described in the context of separate embodiments, may also be provided in combination in a single embodiment. Conversely, various features of the invention that are, for brevity, described in the context of a single embodiment, may also be provided separately or in any suitable sub-combination.

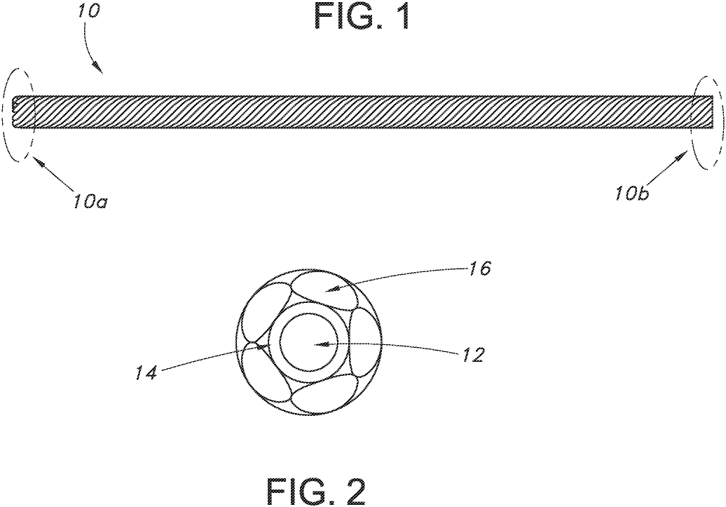

With reference to FIGS. 1-2, a guidewire 10 is shown. As perhaps best understood with reference to the end view of FIG. 2, the guidewire 10 comprises an inner core wire 12, which may comprise a solid piece of material. Over the core wire 12 and at least partially surrounding it is an intermediate layer 14, which as outlined further in the description that follows may comprise a resin tape or resin sleeve extending over the core wire. An outer coil wire 16 is also provided over the intermediate layer 14, and extends fully from one end 10a of the guidewire 10 to the other end 10b.

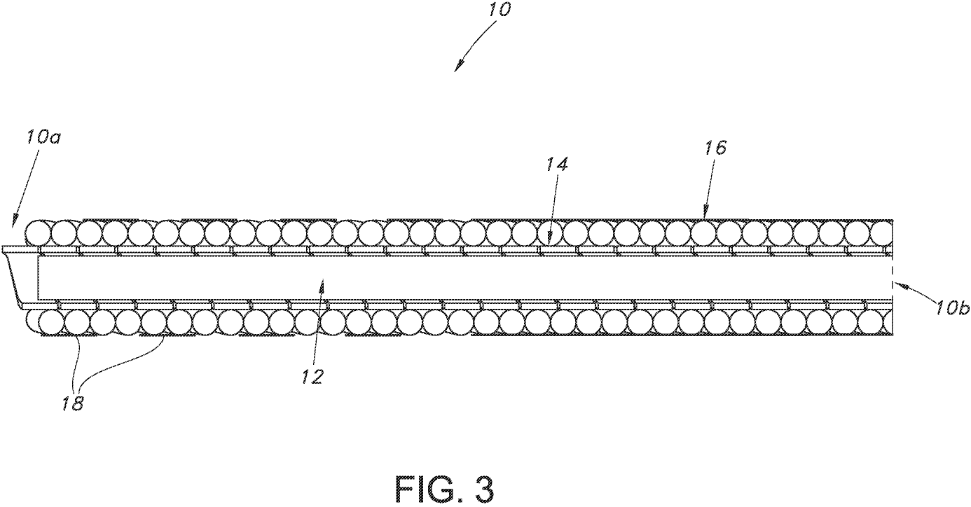

As shown in the partial cross-sectional view of FIG. 3, it can be appreciated that the core wire 12 does not include tapered ends corresponding to the ends of the guidewire 10 (only end 10a is shown, but end 10b would be the same). This means that the core wire 12 is of a substantially constant diameter. Thus, it need not be formed with, or subjected to processing to form, a taper on each end in order to achieve a variable strength profile at the ends, as is characteristic of past approaches, which reduces manufacturing complexity and cost.

As can be further understood, the intermediate layer 14 may comprise a resin tape. This resin tape may be wrapped spirally around the core wire 12. The wrapping may be done in a continuous, but non-overlapping manner, such that each successive wind lies adjacent to the previous one. This is considered to provide the resin tape with a substantially constant pitch.

Once the coil wire 16 is spirally wound around the assembly of the core wire 12, the resin tape serving as the intermediate layer 14 may be subjected to a heat treatment (e.g., indicated by area 18 in FIG. 3) to melt this layer and thus form a bond between the adjacent layers. The heat treatment may be selectively applied, including at spaced locations along the end 10a (and also possibly end 10b) to maintain an enhanced degree of flexibility by only selectively forming a bond between the coil wire 16 and the core wire 12. The selective heat treatment may be achieved using, for example, ultrasonic heating. A weld may also be applied to the ends 10a, 10b to connect the layers forming the guidewire 10 in a secure manner, thereby ensuring the resulting assembly remains intact during repeated use.

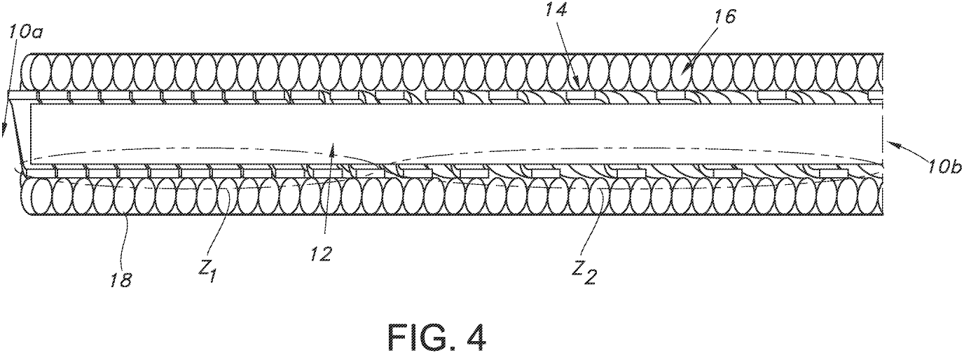

FIG. 4 illustrates an alternative approach in which the intermediate layer 14 comprises resin tape having a variable pitch. Specifically, the pitch of the tape forming this layer 14 is such that successive winds are very close or touching along the end 10a (note zone Z₁), but then gradually increase in spacing in the longitudinal direction (note zone Z₂). The spacing of successive wraps of tape forming the intermediate layer 14 may then decrease again toward the opposite end 10b if desired). Other variations may be applied, depending on the desired strength profile for the guidewire 10.

As indicated, the heat treatment 18 may be continuously applied to the assembled guidewire 10. Again, this may be done using ultrasonic heating, but since it is not selectively applied in this embodiment in view of the variable pitch of the tape forming the intermediate layer 14, an oven could be used instead to apply the heat necessary. In either case, this heating causes the intermediate layer 14 to melt and form a bond. Yet, because of the variable spacing of the tape forming the intermediate layer 14, the flexibility and strength of the guidewire can be adjusted by varying the pitch. This is again achieved without the need for tapering the ends of the core wire 12, which remains of substantially constant diameter.

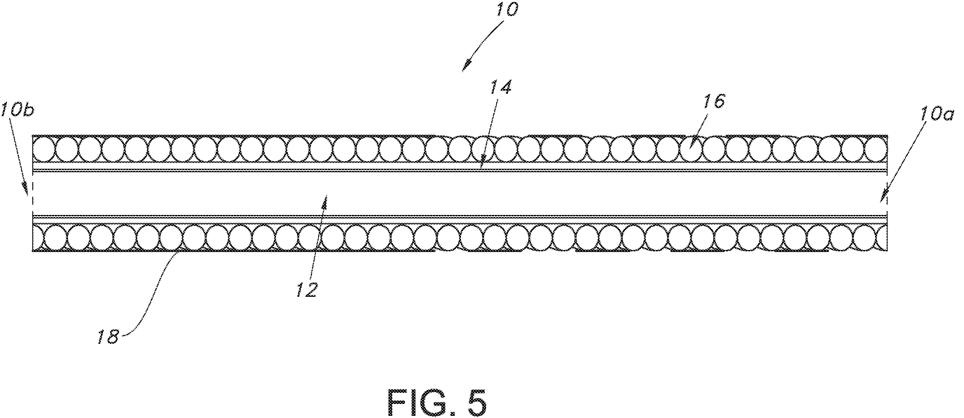

A further embodiment is shown in FIG. 5. In this version, the intermediate layer 14 is formed by a continuous resin sleeve, which may extend along the full length of the guidewire 10 (that is, from one end 10a to the other end 10b). To achieve the variable strength, the heat treatment 18 may be selectively applied. For example, ultrasonic heating may be applied to only spaced portions of the end 10a as indicated, continuously along an intermediate portion of the guidewire 10, and then also selectively at the other end 10b (heat treatment at end 10b not shown). This causes corresponding portions of the intermediate layer 14 to melt and form a bond. Again, a weld may be applied to the ends 10a, 10b to connect the layers in a secure manner.

The guidewire 10 and, in particular, the core wire 12 and the coil wire 16, may be fabricated of commonly used materials such as stainless steel (e.g., 304). The resin tape or sleeve forming intermediate layer 14 may also be formed of a polymer material, such as thermoplastic polyurethane (TPU) or ethylene-vinyl acetate copolymer (EVA). One or more coating or jacketing layers may also optionally be provided, which may comprise polymer materials.

Summarizing, this disclosure may be considered to relate to the following items:
1. A guidewire, comprising:
   a core wire;
   a resin layer at least partially surrounding the core wire; and
   a coil wire over or external to the resin layer.
2. The guidewire of item 1, wherein the core wire has a substantially constant diameter.
3. The guidewire of item 1 or item 2, wherein the resin layer comprises a spirally wound tape.
4. The guidewire of item 3, wherein the spirally wound tape comprises windings having a generally constant pitch.
5. The guidewire of item 3, wherein the spirally wound tape comprises windings having a variable pitch including first windings having a first spacing at a first end of the guidewire and second windings having a second spacing away from the first end of the guidewire, and optionally the second spacing is greater than the first spacing.
6. The guidewire of item 1 or item 2, wherein the resin layer comprises a sleeve.
7. The guidewire of any of items 1-6, wherein the resin layer is melted so as to form a bond between the core wire and the coil wire.
8. The guidewire of any of items 1-7, wherein the resin layer is melted at selected or spaced locations along the guidewire so as to form a bond between the core wire and the coil wire.
9. The guidewire of any of items 1-8, further comprising welded ends.
10. A guidewire, comprising:
   a core wire;
   a resin sleeve at least partially surrounding the core wire; and
   a coil wire over or external to the resin sleeve.
11. The guidewire of item 10, wherein the core wire has a substantially constant diameter.
12. The guidewire of item 10 or item 11, wherein the resin sleeve is melted at selected locations so as to form a bond between the core wire and the coil wire.
13. A guidewire, comprising:
   a core wire;
   a resin tape at least partially surrounding the core wire; and
   a coil wire over or external to the resin tape.
14. The guidewire of item 13, wherein the core wire has a substantially constant diameter.
15. The guidewire of item 13 or item 14, wherein the resin tape is spirally wound around the core wire.
16. The guidewire of item 15, wherein the spirally wound resin tape comprises windings having a substantially constant pitch.
17. The guidewire of item 15, wherein the spirally wound resin tape comprises windings having a variable pitch including first windings having a first spacing at a first end of the guidewire and second windings having a second spacing away from the first end of the guidewire, and optionally the second spacing is greater than the first spacing.
18. The guidewire of any of items 13-17, wherein the resin tape is melted so as to form a bond between the core wire and the coil wire.
19. The guidewire of any of items 13-18, wherein the resin tape is melted at selected or spaced locations along the guidewire so as to form a bond between the core wire and the coil wire.
20. A method of forming a guidewire, comprising:
   providing a resin layer between an inner core wire and an outer coil wire;
   applying heat to at least partially melt the resin layer and bond the inner core wire to the outer coil wire.
21. The method of item 20, wherein the providing step comprises wrapping a resin tape around the inner core wire.
22. The method of item 21, wherein the wrapping is done at a variable pitch.
23. The method of item 21, wherein the wrapping is done at a substantially constant pitch.
24. The method of any of items 20-23, wherein the applying step comprises applying the heat selectively to melt only portions of the resin layer, such as at spaced locations along the guidewire.
25. The method of item 20 or item 24, wherein the providing step comprises providing a resin sleeve over the inner core wire.

Although the invention has been described in conjunction with specific embodiments, many alternatives, modifications, and variations will be apparent to those skilled in the art. Accordingly, it embraces all such alternatives, modifications, and variations that fall within the appended claims’ spirit and scope. It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub-combination in the context or format of a single embodiment may also be illustratively described and presented in the context or format of a plurality of separate embodiments.

All publications, patents and patent applications mentioned in this specification are herein incorporated in their entirety by reference into the specification, to the same extent as if each individual publication, patent or patent application was specifically and individually indicated to be incorporated herein by reference. In addition, the citation or identification of any reference in this application shall not be construed as an admission that such reference is available as prior art to the present disclosure.

1. A guidewire, comprising:
a core wire;
a resin layer at least partially surrounding the core wire; and
a coil wire external to the resin layer.

2. The guidewire of claim 1, wherein the core wire has a substantially constant diameter.

3. The guidewire of claim 1, wherein the resin layer comprises a spirally wound tape.

4. The guidewire of claim 3, wherein the spirally wound tape comprises windings having a generally constant pitch.

5. The guidewire of claim 3, wherein the spirally wound tape comprises windings having a variable pitch including first windings having a first spacing at a first end of the guidewire and second windings having a second spacing away from the first end of the guidewire.

6. The guidewire of claim 5, wherein the second spacing is greater than the first spacing.

7. The guidewire of claim 1, wherein the resin layer comprises a sleeve.

8. The guidewire of claim 1, wherein the resin layer is melted so as to form a bond between the core wire and the coil wire.

9. The guidewire of claim 1, wherein the resin layer is melted at locations spaced along the length of the guidewire so as to form a bond between the core wire and the coil wire.

10. The guidewire of claim 1, further comprising welded ends.

11. A guidewire, comprising:
a core wire;
a resin sleeve at least partially surrounding the core wire; and
a coil wire external to the resin sleeve.

12. The guidewire of claim 10, wherein the core wire has a substantially constant diameter.

13. The guidewire of claim 10, wherein the resin sleeve is melted at locations spaced along the length of the guidewire so as to form a bond between the core wire and the coil wire.

14. A guidewire, comprising:
a core wire;
a resin tape at least partially surrounding the core wire; and
a coil wire external to the resin tape.

15. The guidewire of claim 14, wherein the core wire has a substantially constant diameter.

16. The guidewire of claim 14, wherein the resin tape is spirally wound around the core wire.

17. The guidewire of claim 16, wherein the spirally wound resin tape comprises windings having a substantially constant pitch.

18. The guidewire of claim 15, wherein the spirally wound resin tape comprises windings having a variable pitch including first windings having a first spacing at a first end of the guidewire and second windings having a second spacing away from the first end of the guidewire.

19. The guidewire of claim 18, wherein the second spacing is greater than the first spacing.

20. The guidewire of claim 14, wherein the resin tape is melted so as to form a bond between the core wire and the coil wire.

21. The guidewire of claim 14, wherein the resin tape is melted at locations spaced along the guidewire so as to form a bond between the core wire and the coil wire.

22. A method of forming a guidewire, comprising:
providing a resin layer between an inner core wire and an outer coil wire;
applying heat to at least partially melt the resin layer and bond the inner core wire to the outer coil wire.

23. The method of claim 22, wherein the providing step comprises wrapping a resin tape around the inner core wire.

24. The method of claim 23, wherein the wrapping is done at a variable pitch.

25. The method of claim 24, wherein the wrapping is done at a substantially constant pitch.

26. The method of claim 22, wherein the applying step comprises applying the heat selectively to melt only portions of the resin layer. spaced along the length of the guidewire.

27. The method of claim 22, wherein the providing step comprises providing a resin sleeve over the inner core wire.
